(19) United States
(12) Patent Application Publication
(10) Pub. No.: US 2013/0079574 A1
(43) Pub. Date: Mar. 28, 2013

(54) OLIGOMERIZATION PROCESS

(71) Applicants: Charles P. Luebke, Mount Prospect, IL (US); Steven Lee Krupa, Fox River Grove, IL (US); Robert L. Mehlberg, Wheaton, IL (US)

(72) Inventors: Charles P. Luebke, Mount Prospect, IL (US); Steven Lee Krupa, Fox River Grove, IL (US); Robert L. Mehlberg, Wheaton, IL (US)

(73) Assignee: UOP, LLC., Des Plaines, IL (US)

(21) Appl. No.: 13/243,425

(22) Filed: Sep. 23, 2011

(51) Int. Cl. C07C 2/02

(52) U.S. Cl. 585/503; 585/504

(57) ABSTRACT

One exemplary embodiment can be a process for oligomerizing one or more hydrocarbons. The process can include oligomerizing a feed including one or more C3-C5 hydrocarbons to produce an effluent, and recycling at least a portion of the effluent for oligomerizing. Typically, the recycled portion has at least about 50%, by weight, one or more alkenes based on the weight of the recycled portion.

### FIELD OF THE INVENTION

This invention generally relates to an oligomerization process.

### DESCRIPTION OF THE RELATED ART

Increasing the propene production in a fluid catalytic cracking (hereinafter may be abbreviated as FCC) unit can require the recycle of one or more C4⁺ alkenes back to one or more risers in the fluid catalytic cracking unit for further propene production. However, one shortcoming is that the increased recycle rate to the FCC reactor can accumulate alkanes in this recycle stream. As a result, practically inert alkanes may occupy capacity in the reactor section as well as the product recovery section. This unnecessary recycling of unreactive material can increase the cost and utilities of the process unit. As a consequence, there is a desire to develop alternative schemes for removing this shortcoming by incorporating an FCC process with other units.

### SUMMARY OF THE INVENTION

One exemplary embodiment can be a process for oligomerizing one or more hydrocarbons. The process can include oligomerizing a feed including one or more C3-C5 hydrocarbons to produce an effluent, and recycling at least a portion of the effluent for oligomerizing. Typically, the recycled portion has at least about 50%, by weight, one or more alkenes based on the weight of the recycled portion.

Another exemplary embodiment can be a process for oligomerizing one or more hydrocarbons. Usually, the process includes providing a feed having butene to an oligomerization reaction zone, obtaining from at least a portion of an effluent from the oligomerization reaction zone a first portion and a second portion, recycling the first portion to the oligomerization reaction zone, and sending the second portion to a fluid catalytic cracking zone for producing propene. Usually, each portion has at least about 50%, by weight, one or more alkenes based on the weight of the recycled portion.

A further exemplary embodiment may be a process for oligomerizing one or more hydrocarbons. The process can include providing a feed, obtaining an effluent from the oligomerization reaction zone, passing the effluent to a separation zone to obtain a first stream including butane and a second stream including one or more C5⁺ hydrocarbons, obtaining from the second stream a first portion recycled to the oligomerization reaction zone and a second portion, and sending the second portion to a fluid catalytic cracking zone including a riser reactor operated at a temperature of about 120-about 600° C. for producing propene. Generally, the feed includes butene and the oligomerization reaction zone operates at a temperature of about 120-about 600° C. Usually, the oligomerization reaction zone effluent has an effective amount of one or more alkenes for producing propene in the fluid catalytic cracking zone. In addition, the first portion has at least about 50%, by weight, one or more alkenes based on the weight of the first portion.

The embodiments disclosed herein permit the recycling of one or more C8⁺ alkenes to an FCC zone. A C4 alkene rich stream may be separated in an oligomerization unit and thus may not be recycled back to the FCC zone. As a result, the amount of recycled material may be reduced and the operating costs of the FCC zone can be reduced as well.

Generally, an oligomerization unit can be designed to produce a saturated gasoline product with a limited amount, typically less than about 10%, of one or more C12⁺ hydrocarbons. An alkene product is generally desired and the relative amounts of C8 and C12 alkenes generally do not negatively impact FCC operation. The configuration can be modified so that traditional units such as a saturation reactor, hydrogen addition, stripper column, and rerun column in an oligomerization unit can be eliminated. Thus, capital and operating costs may be reduced.

### DEFINITIONS

As used herein, the term “stream” can be a stream including various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C1, C2, C3 . . . Cn where “n” represents the number of carbon atoms in the one or more hydrocarbon molecules.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “rich” can mean an amount of generally at least about 50%, and preferably about 70%, by mole, of a compound or class of compounds in a stream.

As used herein, the term “substantially” can mean an amount of generally at least about 80%, preferably about 90%, and optimally about 99%, by mole, of a compound or class of compounds in a stream.

As used herein, the term “riser reactor” generally means a reactor used in a fluid catalytic cracking process that can include a riser and a reaction vessel. Usually, such a riser reactor operates by providing catalyst at the bottom of a riser that proceeds to a reaction vessel having a mechanism for separating the catalyst from a hydrocarbon.

As used herein, the terms “alkanes” and “paraffins” may be used interchangeably.

As used herein, the terms “alkenes” and “olefins” may be used interchangeably.

As used herein, the term “weight percent” may be abbreviated as “wt. %”.

As used herein, the process flow lines in the figures can be referred to interchangeably as, e.g., lines, feeds, mixtures, effluents, portions, parts, products, or streams.

### BRIEF DESCRIPTION OF THE DRAWINGS

FIG. 1 is a schematic depiction of an exemplary oligomerization unit.

FIG. 2 is a schematic depiction of an exemplary FCC zone.

### DETAILED DESCRIPTION

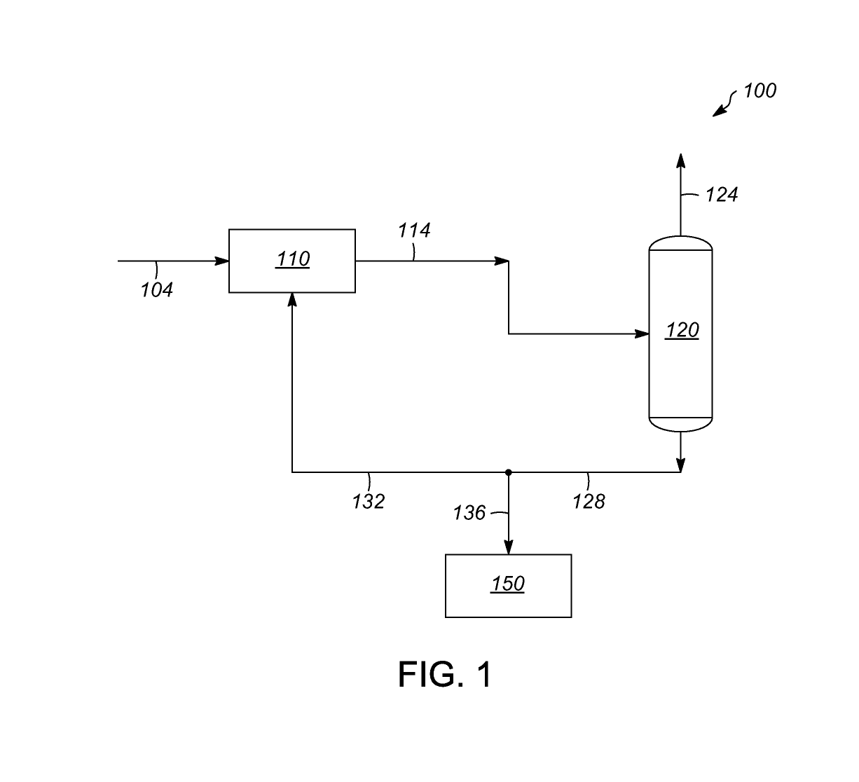

Referring to FIG. 1, an exemplary oligomerization unit 100 can include an oligomerization reaction zone 110, a separation zone 120, and an FCC zone 150. Generally, an oligomerization feed 104 including one or more C3-C5 hydrocarbons can be provided to the oligomerization reaction zone 110. Usually, the oligomerization feed 104 can include butene and butane.

Typically, the oligomerization reaction zone 110 can be operated at any suitable conditions to form one or more C8⁺ hydrocarbons, such as oligomerized C3-C5 hydrocarbons, such as C8, C9, C12, C16 and C20 alkenes. One exemplary oligomerization reaction zone 110 can be operated at a temperature of about 30-about 260° C. and a pressure of about 790-about 8,400 kPa. Usually, the oligomerization reaction zone 110 can receive a recycled portion 132, as hereinafter described, in addition to the oligomerization feed 104. Exemplary oligomerization reaction zones are disclosed in, e.g., U.S. Pat. No. 5,895,830.

The oligomerization reaction zone 110 can produce an oligomerization effluent 114. Generally, the oligomerization effluent 114 has an effective amount of one or more alkenes, preferably one or more C9⁺ alkenes, for producing propene in the FCC zone 150. Often, the oligomerization effluent 114 includes one or more C8⁺ hydrocarbons, preferably one or more C9⁺ hydrocarbons, generally including a rich amount of alkenes. Usually, the oligomerization effluent 114 can have at least about 50% by weight, one or more C9⁺ alkenes based on the weight of the oligomerization effluent 114, and the one or more alkenes or C9⁺ alkenes can include nonene, decene, undecene, and dodecene. Moreover, the oligomerization effluent 114 may include one or more C16⁺ hydrocarbons, such as C16 and C20 hydrocarbons. Generally, the oligomerization effluent 114 can include at least generally about 50%, preferably about 90%, by weight, one or more alkenes based on the weight of the oligomerization effluent 114. The composition of the oligomerization effluent 114 is usually dependent on the oligomerization feed 104, and may be lower than about 50%, by weight, of one or more alkenes, based on the weight of the oligomerization effluent 114. This lower alkene amount may result due to significant amount of alkanes in the oligomerization feed 104.

The oligomerization effluent 114 can be provided to the separation zone 120. The separation zone 120 can include any suitable separation device, such as a distillation column, operating at any suitable temperature to separate a first stream or stream 124 including one or more C4⁻ hydrocarbons, typically butane, and a second stream 128 including one or more C5⁺ hydrocarbons. In other exemplary embodiments, the separation zone 120 may include other devices in addition or instead of a distillation column, such as a flash drum.

Generally, the second stream 128 can be split into the recycled or first portion 132 and another or second portion 136. The recycled portion 132 can have a similar composition as the oligomerization effluent 114, minus the one or more C4⁻ hydrocarbons. Typically, the recycled portion 132 includes at least about 50% by weight, one or more C9⁺ alkenes based on the weight of the recycled portion 132, which can include nonene, decene, undecene, and dodecene. Moreover, the recycled portion 132 may include at least generally about 50%, preferably about 90%, and optimally about 95%, by weight, one or more alkenes based on the weight of the recycled portion 132. This recycled portion 132 can be provided back to the oligomerization reaction zone 110 and provide a quench to permit the operation of the oligomerization reaction zone 110 in a liquid phase and provide benefits of, e.g., extended catalyst life. The another or second portion 136 can be provided to the FCC zone 150 for obtaining propene. Desirably, the second portion 136 includes one or more C9, C12, and C16 hydrocarbons for the production of lighter alkenes, such as propene.

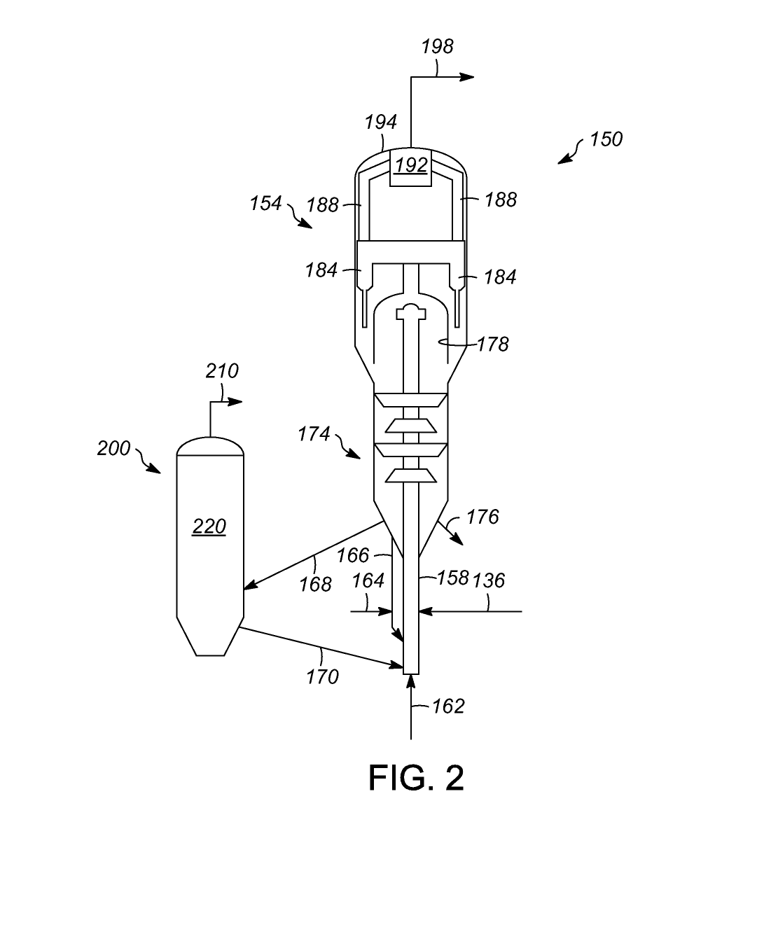

Referring to FIG. 2, the FCC zone 150 can include a riser reactor 154 and a regeneration zone 200. Although only one riser reactor 154 is depicted, it should be understood that multiple riser reactors can be provided, and in one exemplary embodiment two riser reactors can be communicated with a regeneration zone. Generally, the second portion 136 is provided to a riser 158 of the riser reactor 154. If a dual riser reactor zone is utilized, it should be understood that one riser can process a heavier or asphalted hydrocarbon feed while the second riser can process a lighter feed, such as the second portion 136. Such dual riser systems are disclosed in, e.g., US 2010/0236980 A1. The FCC zone 150 can be incorporated into the oligomerization unit 100, or be part of an FCC unit.

The riser reactor 154 can include the riser 158 terminating in a shell 178 housed in a reaction vessel 194. The riser 158 can receive the second portion 136 that optionally may be combined with other hydrocarbons, such as hydrocarbons having a boiling point range of about 180-about 800° C., such as at least one of a gas oil, a vacuum gas oil, an atmospheric gas oil, an atmospheric residue, a heavy cycle oil, and a slurry oil.

Generally, the second portion 136 can be provided at any suitable height on the riser 158, such as above a line 162 providing a lift gas, such as steam and/or a light hydrocarbon, to the riser 158. The second portion 136 may be provided at a distance sufficient to provide a good dispersion of the up-flowing feed and/or catalyst, if desired.

The catalyst can be a single catalyst or a mixture of different catalysts. Such a catalyst mixture is disclosed in, e.g., U.S. Pat. No. 7,312,370 B2 and US 2010/0236980 A1. Alternatively, a single catalyst may be used, such as a medium or smaller pore zeolite catalyst, such as an MFI zeolite, as exemplified by at least one of ZSM-5, ZSM-11, ZSM-12, ZSM-23, ZSM-35, ZSM-38, ZSM-48, and other similar materials. Other suitable medium or smaller pore zeolites include ferrierite, and erionite. Preferably, the medium or smaller pore zeolite dispersed on a matrix including a binder material such as silica or alumina and an inert filler material such as kaolin. The catalyst may also include some other active material such as Beta zeolite. These compositions may have a crystalline zeolite content of about 10-about 50 weight percent or more, and a matrix material content of about 50-about 90 wt. %. Generally, medium and smaller pore zeolites are characterized by having an effective pore opening diameter of less than or equal to about 0.7 nm, rings of about 10 or fewer members, and a Pore Size Index of less than about 31.

Generally, the second portion 136 and the catalyst mixture can be provided proximate to the bottom of the riser 158. Typically, the riser 158 operates with dilute phase conditions above the point of feed injection with a density that is less than about 320 kg/m³. Usually, the second portion 136 is introduced into the riser 158 by a nozzle, and has a temperature of about 140-about 320° C. Moreover, additional amounts of feed may also be introduced upstream or downstream of the initial feed point.

Also, the riser reactor 154 in one desired embodiment can be operated at low hydrocarbon partial pressure. Generally, a low hydrocarbon partial pressure can facilitate the production of light alkenes. Accordingly, the riser 158 pressure can be about 170-about 250 kPa, with a hydrocarbon partial pressure of about 35-about 180 kPa, preferably about 70-about 140 kPa. A relatively low partial pressure for hydrocarbon may be achieved by using steam as a diluent, in the amount of about 10-about 55 wt. %, preferably about 15 wt. %, based on the feed. Other diluents, such as dry gas, can be used to reach equivalent hydrocarbon partial pressures.

The one or more hydrocarbons and catalyst rise to the shell 178 converting the second portion 136. Usually, the second portion 136 reacts within the riser 158 to form one or more products. The riser 158 can operate at any suitable temperature, and typically operates at a temperature of about 150-about 580° C., preferably about 520-about 580° C. In one exemplary embodiment, a higher riser temperature may be desired, such as no less than about 565° C. Exemplary risers are disclosed in, e.g., U.S. Pat. No. 5,154,818 and U.S. Pat. No. 4,090,948.

The products can rise within the riser 158 and exit within the shell 178. Typically, products including propene and gasoline are produced. Subsequently, the catalyst can separate assisted by any suitable device, such as swirl arms, and settle to the bottom of the riser reactor 154. In contrast, one or more products and any remaining entrained catalyst can rise. The entrained catalyst can be separated using separation devices, such as one or more cyclone separators 184 for separating out the products from the catalyst particles. Dip legs may drop the catalyst down to the base of the reaction vessel 194 where openings can permit entry of the spent catalyst into a dense catalyst bed. Exemplary separation devices and swirl arms are disclosed in, e.g., U.S. Pat. No. 7,312,370 B2.

Next, the catalyst can fall to a stripping zone 174. The catalyst may pass through the stripping zone 174 where absorbed hydrocarbons can be removed from the surface of this catalyst by counter-current contact with steam. An exemplary stripping zone is disclosed in, e.g., U.S. Pat. No. 7,312,370 B2. Some catalyst may pass via a line 166, optionally being combined with make-up catalyst in a line 164, and provided at a base of the riser 158 to provide additional heat for facilitating reactions. Also, some catalyst may be removed via a line 176 to maintain catalyst activity.

Afterwards, the catalyst can be regenerated by passing through a line 168 to the regeneration zone 200. The regeneration zone 200 can include a regeneration vessel 220 with flue gas exiting through a line 210. Exemplary regeneration vessels are disclosed in, e.g., U.S. Pat. Nos. 7,312,370 B2 and 7,247,233 B1. The regenerated catalyst can return to the riser 158 via a line 170.

The one or more products leaving the cyclone separators 184 can exit through a plurality of lines 188 before entering a plenum 192 of the reaction vessel 194. Afterwards, a product 198 can pass from the reaction vessel 194 for further processing to, e.g., a product separation zone having one or more distillation columns. Such zones are disclosed in, e.g., U.S. Pat. No. 3,470,084. Usually, the product separation zone may produce several products, such as a propene product and a gasoline product.

The oligomerization reaction zone 110 as described herein can operate in an all liquid phase environment and should still have relatively long catalyst life. Generally, it is expected that the product from this configuration can contain one or more C12⁺ hydrocarbons with some of such hydrocarbons provided to the FCC zone 150. The all liquid phase operation with an alkene recycle can provide greater stability and result in significantly longer catalyst life. Generally, the life expectation for this mode of operation compared to a catalyst polymerization operation may be shorter than an oligomerization unit using an alkane quench due to the higher alkene content of the combined feed to the reactor.

In a further embodiment, an aromatic FCC heavy naphtha can be added to the oligomerization feed 104 to improve solvency of the liquid, and the heavy naphtha can be recovered by distillation at the FCC zone. Further still, a C12⁺ bottom cut of the one or more hydrocarbons can be saturated and recycled back to the oligomerization reaction zone 110. What is more, an octene rich side cut can be obtained from the separation zone 120 and provided to a gasoline pool to meet increased octane requirements or volume requirements.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A process for oligomerizing one or more hydrocarbons, comprising:
A) oligomerizing a feed comprising one or more C3-C5 hydrocarbons to produce an effluent; and
B) recycling at least a portion of the effluent for oligomerizing wherein the recycled portion comprises at least about 50%, by weight, one or more alkenes based on the weight of the recycled portion.

2. The process according to claim 1, wherein the one or more alkenes comprises nonene, decene, undecene, and dodecene.

3. The process according to claim 1, wherein the recycled portion comprises at least about 50%, by weight, one or more C9⁺ alkenes based on the weight of the recycled portion.

4. The process according to claim 1, wherein the recycled portion comprises at least about 90%, by weight, one or more alkenes based on the weight of the recycled portion.

5. The process according to claim 1, further comprising passing the effluent to a separation zone.

6. The process according to claim 5, wherein the separation zone provides a first stream comprising one or more C4⁻ hydrocarbons and a second stream comprising one or more C5⁺ hydrocarbons.

7. The process according to claim 6, wherein the second stream provides the recycled portion and another portion for a fluid catalytic cracking zone.

8. The process according to claim 7, wherein the fluid catalytic cracking zone comprises a riser reactor comprising a riser and the process further comprises providing the another portion to the riser for producing propene.

9. The process according to claim 1, wherein the feed comprises butene and butane.

10. The process according to claim 8, wherein the another portion comprises one or more C12 and C16 hydrocarbons.

11. A process for oligomerizing one or more hydrocarbons, comprising:
A) providing a feed comprising butene to an oligomerization reaction zone;
B) obtaining from at least a portion of an effluent from the oligomerization reaction zone a first portion and a second portion wherein each portion comprises at least about 50%, by weight, one or more alkenes based on the weight of the recycled portion;
C) recycling the first portion to the oligomerization reaction zone; and
D) sending the second portion to a fluid catalytic cracking zone for producing propene.

12. The process according to claim 11, wherein the oligomerization reaction zone is at a temperature of about 30-about 260° C.

13. The process according to claim 11, wherein the oligomerization reaction zone is at a pressure of about 790-about 8,400 kPa.

14. The process according to claim 11, wherein the fluid catalytic cracking zone comprises a riser reactor, and the riser reactor operates at a temperature of about 120-about 600° C.

15. The process according to claim 11, wherein the recycled portion comprises at least about 90%, by weight, one or more alkenes based on the weight of the recycled portion.

16. The process according to claim 11, wherein the recycled portion comprises at least about 95%, by weight, one or more alkenes based on the weight of the recycled portion.

17. The process according to claim 11, further comprising providing the effluent from the oligomerization reaction zone to a separation zone.

18. The process according to claim 17, further comprising obtaining a stream comprising one or more C4⁻ hydrocarbons.

19. The process according to claim 18, wherein the stream comprises butane.

20. A process for oligomerizing one or more hydrocarbons, comprising:
A) providing a feed comprising butene to an oligomerization reaction zone operating at a temperature of about 120-about 600° C.;
B) obtaining an effluent from the oligomerization reaction zone wherein the effluent comprises an effective amount of one or more alkenes for producing propene in a fluid catalytic cracking zone;
C) passing the effluent to a separation zone to obtain a first stream comprising butane and a second stream comprising one or more C5⁺ hydrocarbons;
D) obtaining from the second stream a first portion recycled to the oligomerization reaction zone wherein the first portion comprises at least about 50%, by weight, one or more alkenes based on the weight of the first portion and a second portion; and
E) sending the second portion to the fluid catalytic cracking zone comprising a riser reactor operated at a temperature of about 120-about 600° C. for producing propene.
